SEQUENCE LISTING (38 sequences)

SEQ ID NO: 1 - protein, 16 aa, synthetic construct
  REGION 1..16: note = Synthetic peptide
LGLKLLWSAYKHRKTL

SEQ ID NO: 2 - protein, 16 aa, synthetic construct
  REGION 1..16: note = Synthetic peptide
LTKRHKYASWLLKLGL

SEQ ID NO: 3 - protein, 16 aa, synthetic construct
  REGION 1..16: note = Synthetic peptide
LGIKILWSAYKHRKTI

SEQ ID NO: 4 - protein, 16 aa, synthetic construct
  REGION 1..16: note = Synthetic peptide
ITKRHKYASWLIKIGL

SEQ ID NO: 5 - protein, 34 aa, synthetic construct
  REGION 1..34: note = Synthetic
SVPTSVYTLGIKILWSAYKHRKTIEKSFNKGFYH

SEQ ID NO: 6 - protein, 16 aa, synthetic construct
  REGION 1..16: note = Synthetic peptide
SVPTSVYTLGIKILWS

SEQ ID NO: 7 - protein, 16 aa, synthetic construct
  REGION 1..16: note = Synthetic peptide
VPTSVYTLGIKILWSA

SEQ ID NO: 8 - protein, 16 aa, synthetic construct
  REGION 1..16: note = Synthetic peptide
PTSVYTLGIKILWSAY

SEQ ID NO: 9 - protein, 16 aa, synthetic construct
  REGION 1..16: note = Synthetic peptide
TSVYTLGIKILWSAYK

SEQ ID NO: 10 - protein, 16 aa, synthetic construct
  REGION 1..16: note = Synthetic peptide
SVYTLGIKILWSAYKH

SEQ ID NO: 11 - protein, 16 aa, synthetic construct
  REGION 1..16: note = Synthetic peptide
VYTLGIKILWSAYKHR

SEQ ID NO: 12 - protein, 16 aa, synthetic construct
  REGION 1..16: note = Synthetic peptide
YTLGIKILWSAYKHRK

SEQ ID NO: 13 - protein, 16 aa, synthetic construct
  REGION 1..16: note = Synthetic peptide
TLGIKILWSAYKHRKT

SEQ ID NO: 14 - protein, 16 aa, synthetic construct
  REGION 1..16: note = Synthetic peptide
GIKILWSAYKHRKTIE

SEQ ID NO: 15 - protein, 16 aa, synthetic construct
  REGION 1..16: note = Synthetic peptide
IKILWSAYKHRKTIEK

SEQ ID NO: 16 - protein, 16 aa, synthetic construct
  REGION 1..16: note = Synthetic peptide
KILWSAYKHRKTIEKS

SEQ ID NO: 17 - protein, 16 aa, synthetic construct
  REGION 1..16: note = Synthetic peptide
ILWSAYKHRKTIEKSF

SEQ ID NO: 18 - protein, 16 aa, synthetic construct
  REGION 1..16: note = Synthetic peptide
LWSAYKHRKTIEKSFN

SEQ ID NO: 19 - protein, 16 aa, synthetic construct
  REGION 1..16: note = Synthetic peptide
WSAYKHRKTIEKSFNK

SEQ ID NO: 20 - protein, 16 aa, synthetic construct
  REGION 1..16: note = Synthetic peptide
SAYKHRKTIEKSFNKG

SEQ ID NO: 21 - protein, 16 aa, synthetic construct
  REGION 1..16: note = Synthetic peptide
AYKHRKTIEKSFNKGF

SEQ ID NO: 22 - protein, 16 aa, synthetic construct
  REGION 1..16: note = Synthetic peptide
YKHRKTIEKSFNKGFY

SEQ ID NO: 23 - protein, 16 aa, synthetic construct
  REGION 1..16: note = Synthetic peptide
KHRKTIEKSFNKGFYH

SEQ ID NO: 24 - protein, 18 aa, synthetic construct
  REGION 1..18: note = Synthetic peptide
LGIKILRSAYKHRKTIEK

SEQ ID NO: 25 - protein, 22 aa, synthetic construct
  REGION 1..22: note = Synthetic peptide
LGIKILRSAYKHRKTIEKSFNK

SEQ ID NO: 26 - protein, 20 aa, synthetic construct
  REGION 1..20: note = Synthetic peptide
LGQKILRSARKFGKTIEKSF

SEQ ID NO: 27 - protein, 20 aa, synthetic construct
  REGION 1..20: note = Synthetic peptide
LGQKILRSARKFGKDIEKSF

SEQ ID NO: 28 - protein, 20 aa, synthetic construct
  REGION 1..20: note = Synthetic peptide
LGIKILRSARKFGKVIEKSF

SEQ ID NO: 29 - protein, 20 aa, synthetic construct
  REGION 1..20: note = Synthetic peptide
LGIKILWSARKFGKVIEKSF

SEQ ID NO: 30 - protein, 21 aa, synthetic construct
  REGION 1..21: note = Synthetic peptide
LLGIKILWSARKFGKVIEKSF

SEQ ID NO: 31 - protein, 21 aa, synthetic construct
  REGION 1..21: note = Synthetic peptide
LLGIKILWKARKFGKVIEKSF

SEQ ID NO: 32 - protein, 10 aa, synthetic construct
  REGION 1..10: note = Synthetic peptide
KILWSAYKHR

SEQ ID NO: 33 - protein, 9 aa, synthetic construct
  REGION 1..9: note = Synthetic peptide
ILWSAYKHR

SEQ ID NO: 34 - protein, 8 aa, synthetic construct
  REGION 1..8: note = Synthetic peptide
LWSAYKHR

SEQ ID NO: 35 - protein, 7 aa, synthetic construct
  REGION 1..7: note = Synthetic peptide
WSAYKHR

SEQ ID NO: 36 - protein, 6 aa, synthetic construct
  REGION 1..6: note = Synthetic peptide
WSAYKH

SEQ ID NO: 37 - protein, 5 aa, synthetic construct
  REGION 1..5: note = Synthetic peptide
WSAYK

SEQ ID NO: 38 - protein, 37 aa, synthetic construct
  REGION 1..37: note = Synthetic peptide
LLGDFFRKSKEKIGKEFKRIVQRIKDFLRNLVPRTES

(19) United States
(12) Patent Application Publication
(10) Pub. No.: US 2025/0320261 A1
(43) Pub. Date: Oct. 16, 2025

(54) ANTIBACTERIAL PEPTIDE

(71) Applicant: CURENC AB, Linköping (SE)

(72) Inventors: Torbjörn BENGTSSON, Linköping (SE); Hazem KHALAF, Örebro (SE); Robert SELEGÅRD, Norsholm (SE); Daniel AILI, Linköping (SE)

(73) Assignee: CURENC AB, Linköping (SE)

(21) Appl. No.: 18/705,888

(22) PCT Filed: Oct. 31, 2022
(86) PCT No.: PCT/SE2022/050996

§ 371 Date: Apr. 29, 2024

(30) Foreign Application Priority Data
Oct. 29, 2021 (SE) 2151338-7

(51) Int. Cl. C07K 14/335

(52) U.S. Cl. CPC C07K 14/335

(57) ABSTRACT

The present document is directed to an antimicrobial peptide, pharmaceutical and non-pharmaceutical compositions comprising an amino acid sequence as set forth in SEQ ID NO: 1 or SEQ ID NO: 2 or an amino acid sequence having at least 85% or at least 90%, sequence identity to SEQ ID NO: 1 or SEQ ID NO: 2, and medical and non-medical use the antimicrobial peptide

### TECHNICAL FIELD

The present document is in the field of antimicrobial peptides for medical and non-medical uses.

### BACKGROUND

Antibiotics are the most effective treatment against bacterial infections of both gram-positive (e.g., Staphylococcus aureus) and gram-negative (e.g., Escherichia coli) species. Many species are opportunistic pathogens that may cause severe infections in humans connected to chronic wounds and medical devices, e.g., catheters and prosthetic implants 1. These bacterial accumulations are the basis of persistent infections that are generally difficult to treat, which increases the risk for bacterial dissemination and development of systemic complications^{2,3} Furthermore, considering the gradual increase in antibiotic resistance, treatment may be even more difficult to achieve as the available options become limited⁴. Consequently, new approaches of innovative alternative treatments against bacterial infections are urgently needed in human medicine, and antimicrobial peptides represent one of the promising agents that require more consideration^{5,6}.

Since antibiotics are becoming less effective, antimicrobial peptides have become attractive candidates in human medicine due to their characteristics of displaying low toxicity towards eukaryotic cells and considered safe and harmless to humans and are active against pathogenic bacteria that have acquired resistance to antibiotics^{5,7} These peptides generally consist of short sequences with no secondary structure showing stability against heat and changes in pH, and express bactericidal activity against a wide range of microbes⁸⁻¹⁰ Peptides are in vivo exposed to various physical, chemical and biological conditions¹¹, affecting their activity and bioavailability. It has previously shown that the bacteriocin PLNC8 αβ permeabilizes the gram-negative oral pathogen Porphyromonas gingivalis and counteracts its cytotoxic and immunomodulatory effects on human cells^{12,13}. Furthermore, it has recently been shown that PLNC8 αβ is most effective against bacteria of the genus Staphylococcus, including strains that have acquired resistance to antibiotics, and enhances severalfold the activity of different antibiotics¹⁴.

The most problematic and disease-causing pathogens have been categorized by the World Health Organization (WHO) and constitute Enterococcus faecium, Staphylococcus aureus, Klebsiella pneumoniae, Acinetobacter baumannii, Pseudomonas aeruginosa, and Enterobacter species (ESKAPE). These bacteria have developed multidrug resistance against several antibiotic classes and can form biofilms. WHO has listed the ESKAPE pathogens among its top 12 priority pathogens in urgent need for the development of new antimicrobials. Most of the antibiotics recommended in the Clinical & Laboratory Standards Institute guidelines to act against the ESKAPE pathogens have been removed and very limited novel antibiotics or antibiotic combinations have been added in their place¹⁵. Since infections caused by the ESKAPE pathogens are the most problematic infections in humans, it is important to find alternative treatments consisting of novel antimicrobial compounds. The potency and low toxicity of new and innovative substances may potentially reduce the overall use of antibiotics, and consequently the development and spreading of antimicrobial resistance may be suppressed by using antimicrobial peptides, either alone or in combination with low doses of antibiotics.

An object of the present invention is thus to overcome or at least mitigate one or more of the problems described herein.

### SUMMARY

One or more of the above objects may be achieved by an isolated peptide or a salt thereof, such as a pharmaceutically acceptable salt thereof, said peptide:
(a) comprising an amino acid sequence as set forth in SEQ ID NO: 1 or SEQ ID NO: 2 or an amino acid sequence having at least 85% or at least 90%, sequence identity to SEQ ID NO: 1 or SEQ ID NO: 2; or
(b) consisting of an amino acid sequence as set forth in SEQ ID NO: 1 or SEQ ID NO: 2 or an amino acid sequence having at least 75%, such as at least 80%, at least 85%, or at least 90%, sequence identity to SEQ ID NO: 1 or SEQ ID NO: 2. The peptide may e.g. be selected from the group consisting of SEQ ID NO: 1 to SEQ ID NO: 4, i.e., SEQ ID NO: 1, SEQ ID NO: 2, SEQ ID NO: 3 and SEQ ID NO: 4.

The peptide may comprise at least one amino acid residue being a D amino acid, preferably all amino acid residues being D amino acids.

The peptide may comprise at least one amino acid residue being a L amino acid, preferably all amino acid residues being L amino acids.

The peptide may further comprise a hydrophobic moiety bound to the peptide, such as an acyl group, a hydrophobic amino acid, or a lipid covalently bound to the peptide.

The present document is also directed to a nucleic acid sequence encoding peptide as defined herein, a vector comprising said nucleic acid sequence and/or a cell, such as a bacterial cell, comprising said vector or said nucleic acid sequence.

The present document is also directed to a pharmaceutical or non-pharmaceutical composition comprising a peptide as defined herein, a nucleic acid sequence as defined in herein, a vector as defined herein and/or a cell as defined herein.

The present document is also directed to a medical device, such as a surgical suture, sheet, patch, membrane, hydrogel, coating, dressing and/or carrier material, comprising a peptide as defined herein, a nucleic acid sequence as defined herein, a vector as defined herein, a cell as defined herein and/or a pharmaceutical or non-pharmaceutical composition as defined herein.

The present document is also directed to a peptide as defined herein, a nucleic acid sequence as defined herein, a vector as defined herein, a cell as defined in herein and/or a pharmaceutical composition as defined herein for use as a medicament.

The present document is also directed to a peptide as defined herein, a nucleic acid sequence as defined herein, a vector as defined herein, a cell as defined in herein and/or a pharmaceutical composition as defined herein for use in the treatment and/or prevention of a microbial and/or parasitic infection, an inflammation and/or a biofilm.

The microbial infection referred to herein may be a bacterial, viral and/or fungal infection.

The peptide, nucleic acid sequence, vector, cell and/or pharmaceutical or non-pharmaceutical composition may e.g. be used for disinfecting a wound.

The peptide, nucleic acid sequence, vector, cell and/or pharmaceutical or non-pharmaceutical may e.g. be used in overcoming inherent or acquired resistance of a microorganism to an antibiotic.

The peptide as defined herein may also be used as a non-medical antimicrobial, antiparasitic and/or disinfecting agent and/or for removing and/or preventing the formation of a biofilm on a non-biological subject and/or as a biocide, the biocide being preferably for cleaning and/or disinfecting non-biological objects.

Other features and advantages of the invention will be apparent from the following detailed description, drawings, examples, and from the claims.

### Definitions

In the context of the present document, by a peptide or protein sequence having an amino acid sequence at least, for example 95% identical to a reference sequence, is intended that the sequence is identical to the reference sequence except that the sequence may include a combination of up to 5 changes such as amino acid substitutions, deletions and/or insertions per each 100 amino acids of the reference sequence. In other words, to obtain a peptide or protein sequence having a sequence at least 95% identical to a reference sequence only 5% of changes in amino acid sequence are allowed when directly aligned. These changes can be point mutations, substitutions, deletions and/or insertions.

These mutations/amino acid changes of the reference sequence may occur at the amino and/or carboxy terminal positions of a reference amino acid sequence or the 5′ and/or 3′ end of a reference nucleic acid sequence or anywhere between those terminal positions, interspersed either individually within the reference sequence or in one or more contiguous groups within the reference sequence.

As a practical matter, whether any particular peptide has a certain identity to a reference sequence can be determined by using conventional computer programs known to the person skilled in the art, such as BLAST (Myers et al. J. Mal. Biol, 125 (3), p. 403-410, 1990) or FASTDB computer program (based on the algorithm of Brutlag et al. (Comp. App. Biosci. (1990)) 6:237-245).

In the context of the present document the terms “a” and “an” refers may refer to a plurality unless it is clear from the context that singularity is intended.

### BRIEF DESCRIPTION OF DRAWINGS

FIG. 1. Characteristics of the optimized peptide sequence #6 (SEQ ID NO: 3). A) The truncated peptide #6 is composed of 43.75% hydrophobic residues, 31.25% basic residues, and 25% neutral residues, with a net positive charge of 4.5. B) The peptide structure was predicted using the PEP-FOLD tool in the RPBS Wed Portal (https://bioserv.rpbs.univ-paris-diderot.fr/index.html, 24). C) Distribution of amino acids in a helical wheel projection and D) hydrophilicity analysis using the Kyte-Doolittle plot.

FIG. 2. Characteristics of the leucine variant of peptide sequence #6. A) All three isoleucine residues of peptide sequence #6 (SEQ ID NO: 3) were replaced by leucine, resulting in a peptide of SEQ ID NO: 1, and the peptide (SEQ ID NO: 1) is composed of 43.75% hydrophobic residues, 31.25% basic residues, and 25% neutral residues, with a net positive charge of 4.5. B) The peptide structure was predicted using the PEP-FOLD tool in the RPBS Wed Portal (https://bioserv.rpbs.univ-paris-diderot.fr/index.html). C) Distribution of amino acids in a helical wheel projection and D) hydrophilicity analysis using the Kyte-Doolittle plot.

FIG. 3. Permeabilization of bacterial membranes. Uptake of Sytox Green by A) S. aureus and B) E. coli was determined after exposure to different concentrations of acetylated peptides for 5 min, scale bar is 200 μm. Conjugation of the peptide with a fatty acid chain of five to eight carbon atoms is efficient at permeabilizing both gram-positive and gram-negative bacterial membranes. The D-enantiomer and leucine variant (SEQ ID NO: 1 with a carbon tail of 5 carbons) of 6-C5-N(SEQ ID NO: 3 with a carbon tail of 5 carbons) were equally as potent as the L-form at permeabilizing S. aureus and E. coli.

FIG. 4. Hemolytic activity of SEQ ID NO 1 and SEQ ID NO 3. The hemolytic activity was determined on human erythrocytes (15% suspended in PBS) after exposure to A) SEQ ID NO: 3 with increasing length of fatty acid chains and full length PLNC8 β, B) L-enantiomers of SEQ ID NO: 3 with a fatty acid chain length of 5, 6, and 7 carbon (L-6-C5-N; L-6-C6-N; L-6-C7-N), and L-enantiomers of SEQ ID NO 1 with a fatty acid chain length of 5, 6, and 7 carbon (L-6-C5-N-Leu; L-6-C6-N-Leu; L-6-C7-N-Leu), C) D-enantiomers of SEQ ID NO: 3 with a fatty acid chain length of 5, 6, and 7 carbon (D-6-C5-N; D-6-C6-N; D-6-C7-N), and D-enantiomers of SEQ ID NO: 1 with a fatty acid chain length of 5, 6, and 7 carbon (D-6-C5-N-Leu; D-6-C6-N-Leu; D-6-C7-N-Leu). Erythrocytes were exposed to the peptides using the indicated concentrations for 1 h. Hemolytic activity was associated with the length of the fatty acid chain, where conjugation of long fatty acid chains (≥7 carbon atoms) rendered the peptides to be more hemolytic. Substitution of all three isoleucine residues with leucine rendered the lipopeptide to be more hemolytic.

FIG. 5. Cytotoxicity and cytokine secretion of infected HaCaT keratinocytes.

After 24 hours of exposure to Staphylococcus aureus (MOI=0.1), and three administrations of L-6-C5-N (2.5, 5, 10, 20 and 40 μM) at 1, 5 and 9 hours post infection. (A) Cytotoxicity, LDH levels normalized against negative control. (B) Interleukin-6. (C) Tumor Necrosis Factor-α. (D) CXC motif ligand 8 levels. (−) indicates negative control (uninfected/unstimulated cells), (+) indicates positive control (S. aureus-infected cells). Statistical analysis was done by ANOVA with Tukey's post hoc test. P-values are indicated by *=0.5, ***=0.001.

FIG. 6. Cytotoxicity and cytokine secretion of uninfected HaCaT keratinocytes.

After 24 hours of exposure to three administrations of L-6-C5-N (2.5, 5, 10, 20 and 40 μM) at 0, 4 and 8 hours. (A) Cytotoxicity, LDH levels normalized against negative control. (B) Interleukin-6. (C) Tumor Necrosis Factor-α. (D) CXC motif ligand 8 levels. (−) indicates negative control. Statistical analysis was done by ANOVA with Tukey's post hoc test. P-values are indicated by *=0.5, ***=0.001.

### DETAILED DESCRIPTION

### Peptides

The present document is directed to a peptide according to SEQ ID NO: 1 or SEQ ID NO: 2 and peptides having a defined identity thereto and which have substantially the same activity as a peptide of SEQ ID NO: 1 or SEQ ID NO: 2. The activity of a peptide of the present document may e.g. be a biological activity, such as an antimicrobial, including antibacterial, anti-parasitic and/or anti-inflammatory activity.

The peptide of the present document is an isolated peptide or a salt thereof, such as a pharmaceutically acceptable salt thereof, wherein said peptide either comprises an amino acid sequence as set forth in SEQ ID NO: 1 or SEQ ID NO: 2 or a peptide having at least 85%, such as at least 90%, sequence identity to SEQ ID NO: 1 or SEQ ID NO: 2 or wherein said peptide consists of an amino acid sequence as set forth in SEQ ID NO: 1 or SEQ ID NO: 2 or a peptide having at least 75%, such as at least 80%, at least 85%, or at least 90%, sequence identity to SEQ ID NO: 1 or SEQ ID NO: 2. The peptide of the present document may be an artificial peptide in the sense that the amino acid sequence of the peptide does not exist as such nature. In the context of the present document, whenever it is referred to a peptide or the peptide of the present document and the like, this is intended to cover also peptides having the herein specified identities to SEQ ID NO: 1 or SEQ ID NO: 2.

L. plantarum NC8 is a bacterium that has previously been shown to produce a two-peptide bacteriocin, PLNC8 αβ, which is classified as a class IIb bacteriocin.

According to the present document, the PLNC8 β sequence of the bacteriocin PLNC8 αβ was used to design new and optimized antimicrobial peptides. The sequence of PLNC8 β was truncated to 16 amino acids (residues #1-16), after which one amino acid was removed from the N-terminal region and one amino acid added to the C-terminal region. This was completed throughout the entire sequence of PLNC8 β, which is composed of 34 amino acids, and resulted in a total of 19 truncated peptides. The antimicrobial activity of full length and truncated peptides of PLNC8 β was predicted using the servers AntiBPs (* https: i/webs.iiitd.edu.in/raghava/antibp2/submit.html) and ADAM (#http://bioinformatics.cs.ntou.edu.tw/adam/svm predict.php). The sequence with the highest score of predicted antimicrobial activity was used to generate 14 additional modified variants. Peptide characteristics, such as molecular weight, chemical formula, iso-electric point, net charge at pH 7, and percentage of hydrophobic, acidic, basic and neutral residues were determined using Peptide 2.0 (https://www.peptide2.com/). Peptide structures were predicted using the PEP-FOLD tool in the RPBS Web Portal (https://bioserv.rpbs.univ-paris-diderot.fr/index.html).

It was surprisingly found that by using only 16 amino acids from a specific part of the β peptide of the bacteriocin PLNC8 αβ, peptides with a strong antimicrobial activity could be provided (SEQ ID NO: 3). Further, replacing the isoleucines of SEQ ID NO: 3 with leucines, also resulted in a peptide, SEQ ID NO: 1, with a strong antimicrobial activity as demonstrated in the experimental section. SEQ ID NO: 2 is the reverse sequence of SEQ ID NO: 1 and SEQ ID NO: 4 is the reverse sequence of SEQ ID NO: 3. The biological activity of SEQ ID NO: 1 and SEQ ID NO: 3, and peptides having certain degrees of identity to SEQ ID NO: 1, was demonstrated as is shown elsewhere herein.

Non-limiting examples of peptides of the present document comprise a peptide selected from the group consisting of SEQ ID NO: 1, SEQ ID NO: 2, SEQ ID NO: 3 and SEQ ID NO: 4.

Preferably, a peptide of the present document is a peptide comprising or consisting of a peptide according to SEQ ID NO: 1 or SEQ ID NO: 2.

The peptides of the present document are preferably isolated or purified. Also, the peptides may be artificial in the sense that they do not exist naturally as such in nature. The peptides of the present document may exist in different forms, such as free acids, free bases, esters, pro-drug forms, salts and tautomers. A peptide of the present document may e.g. be present as a pharmaceutical or a non-pharmaceutical salt.

A pharmaceutically acceptable salt of a peptide of the present document include, but is not limited to, addition salts such as a acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, oxalate, palmoate, pectinate, persulfate, 3-phenylpropionate, picrate, pivalate, propionate, succinate, tartrate, thiocyanate, tosylate, and undecanoate; base salts such as ammonium salts, alkali metal salts such as sodium and potassium salts; alkaline earth metal salts such as calcium and magnesium salts; salts with organic bases such as dicyclohexylamine salts, N-methyl-D-glutamine; and salts with amino acids such as arginine, lysine, etc. Also, basic nitrogen-containing groups may be quaternized with e.g. lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides.

The peptides of the present document may comprise a mix of both D and L amino acids or just D or L amino acids. Preferably, the peptide consists of only D or L amino acids. Peptides comprising or consisting of D amino acids are more stable and less sensitive to proteolytic cleavage compared to their corresponding variants. Thereby, the peptide's biological effect(s) may be prolonged.

The peptides of the present document encompasses peptides according to SEQ ID NO: 1 or SEQ ID NO: 2, a peptide sequence comprising an amino acid sequence having at least 85% identity to SEQ ID NO: 1 or SEQ ID NO: 2, and a peptide consisting of an amino acid sequence having at least 75% identity to SEQ ID NO: 1 or SEQ ID NO: 2. Peptides having a specified identity to SEQ ID NO: 1 or SEQ ID NO: 2 may comprise one or more of a substitution, deletion and/or insertion and/or conservative amino acid substitutions and/or conservatively modified sequence variants.

A peptide comprising an amino acid sequence having at least 85% identity to SEQ ID NO: 1 or SEQ ID NO: 2 may be constructed by substituting, deleting and/or adding one or two amino acids in the amino acid sequence of SEQ ID NO: 1 or SEQ ID NO: 2, i.e. resulting in a peptide having 87.5% identity or 93.75% identity thereto. These substitutions, deletions and/or additions may take place anywhere in the amino acid sequence, i.e. at the C- or N-terminal of the peptide or anywhere in between.

A peptide consisting of an amino acid sequence having at least 75% identity to SEQ ID NO: 1 or SEQ ID NO: 2 may be constructed by substituting, deleting and/or adding one, two, three or four amino acids in the amino acid sequence of SEQ ID NO: 1 or SEQ ID NO: 2 (i.e. resulting in a peptide having 75%, 81.25%, 87.5% identity or 93.75% identity thereto). These substitutions, deletions and/or additions may take place anywhere in the amino acid sequence, i.e. at the C- or N-terminal of the peptide or anywhere in between.

When substituting one or more amino acids in a peptide according to SEQ ID NO: 1 or SEQ ID NO: 2, preferably an amino acid is replaced by an amino acid in the same group, i.e. a hydrophobic amino acid is replaced by another hydrophobic amino acid, a hydrophilic with a hydrophilic etc. Non-limiting examples of such groups of amino acids are the hydrophobic group, comprising the amino acid Ala, Val, Phe, Pro, Leu, Ile, Trp, Met and Cys; the basic group, comprising the amino acid Lys, Arg and His; the hydrophilic group, comprising the uncharged amino acid residues Gin, Asn, Ser, Thr and Tyr; and the neutral group, comprising the amino acid residue Gly.

Importantly, when substituting, deleting or adding amino acids to the peptides of SEQ ID NO: 1 or SEQ ID NO: 2, the resulting peptide should have substantially the same activity, such as a biological activity, such as an antimicrobial, antiparasitic and/or anti-inflammatory activity, as a peptide of SEQ ID NO: 1 or SEQ ID NO: 2. Such an activity can e.g. be tested by testing the antimicrobial activity as described in the experimental section below. Also, preferably the peptides of the present document do not have a significant hemolytic activity if they are to be used for medical purposes.

The positively charged amino acids of the peptide of the present document are necessary for the peptide to be attracted to negatively charged surfaces, such as bacterial membranes, through electrostatic interactions with negatively charged molecules. The hydrophobic amino acids in the peptide for interactions with the membrane leading to the permeabilization. Regarding the whole peptide, an amphiphilic structure is needed to penetrate the membrane. This means, hydrophobic amino acids should preferably only be replaced by hydrophobic amino acids, positively charged amino acids by positively charged amino acids, etc. If amino acids are added or deleted, the amphiphilic structure has to stay intact.

The position and percentage of hydrophobic and positively charged amino acids may also affect toxicity levels, so it is important to apply the above-described pattern when modifying a peptide of SEQ ID NO: 1 or SEQ ID NO: 2.

The peptide may comprise a hydrophobic moiety covalently bound to the peptide, such as an acyl group, a hydrophobic amino acid, or a lipid. Such a hydrophobic group may enhance the activity of a peptide of the present document as is demonstrated in the experimental section. Adding a hydrophobic moiety to the peptide increases the peptide's hydrophobicity. A hydrophobic moiety may thus make the peptide more effective.

The hydrophobic moiety is covalently bound to the hydrophobic part of the peptide. This means that the hydrophobic moiety is attached to the N-terminal end of the peptide if a peptide of SEQ ID NO: 1 or a peptide having the herein specified identity thereto is used. If the peptide used is based on SEQ ID NO: 2, which is the mirror sequence of SEQ ID NO: 1, or a sequence having a specified identity thereto is used, then the hydrophobic moiety is covalently bound to the C-terminal.

It was surprisingly found that by coupling a hydrophobic moiety in the form of an acyl group to a peptide of the present document, the antibacterial activity of the peptide was increased. The acyl group may comprise a carbon chain of a length of at least one carbon, such as from 1 to 15 carbons, from 1 to 12 carbons, from 1 to 10 carbons, preferably at least 2 carbons, such as from 2 to 8 carbons, from 5 to 8 carbons or from 5 to 7 carbons, such as 1, 2, 3, 4, 5, 6, 7, 8, 9, or 10 carbons. The acyl group may e.g. be selected from the group consisting of acetyl, propionyl, butyryl, pentanoyl, hexanoyl, heptanoyl, octanoyl and decanoyl, preferably pentanoyl, hexanoyl, heptanoyl.

Particularly good results were surprisingly obtained when using a carbon chain length of 5 to 8 carbons, which is shorter than what was predicted to give the best results. The coupling of an acyl group to a peptide of the present document may e.g. be performed by carbodiimide chemistry as is well-known to the person skilled in the art and further explained in the experimental section.

A peptide as disclosed herein may be part of a longer peptide, such as a peptide up to about 100 amino acids long, such as about 34 amino acids long, such as about 26 amino acids long or about 20 amino acids long, wherein the peptide of the present document is flanked either in the N-terminal or C-terminal end or both the N-terminal and the C-terminal end with other amino acids.

For example, a peptide of the present document may comprise an N- or C-terminal flanking peptide comprising or consisting of L amino acids. For example, a peptide as defined herein may comprise or consist of D amino acids and be flanked by a covalently bound peptide comprising or consisting of L amino acids, which is degraded to produce a peptide as defined herein. In such a case the flanking peptide preferably consists of L amino acids and the peptide as defined herein consists of D amino acids. If the peptide comprises a covalently bound hydrophobic moiety as explained elsewhere herein, the flanking peptide is positioned on the terminus opposite of the terminus where the hydrophobic moiety is positioned.

The peptide of the present document may also comprise or consist of 12, 13, 14 or 15 of the amino acids of SEQ ID NO: 1 or SEQ ID NO: 2.

Accordingly, a peptide of the present document may be from about 12 to about 100 amino acids, such as from about 12 to about 34 amino acids, such as from about 16 to about 26 amino acids or from about 16 to about 20 amino acids.

A peptide of the present document may also be provided as a pro-peptide comprising a peptide as defined herein, wherein a peptide as defined herein is produced by degradation of said pro-peptide. The pro-peptide may e.g. comprise a stretch of L amino acids, in particular when the peptide comprises or consists of D amino acids.

A peptide of the present document has an antimicrobial, antiparasitic and/or anti-inflammatory activity. The antimicrobial activity may be an antibacterial, antiviral and/or antifungal activity.

A peptide of the present document may have an antimicrobial activity against one or more Gram-negative and/or Gram-positive bacteria. Such gram-negative or gram-positive bacteria may be selected from the group consisting of Staphylococcus spp (including MRSA, MRSE), Streptococcus spp (e.g. S. mutans, S. constellatus, S. anginosus), Enterococcus faecium (including VRE), Klebsiella pneumoniae, Acinetobacter baumannii, Pseudomonas aeruginosa, Enterobacter spp, Escherichia coli, Helicobacter pylori, Campylobacter, Salmonella, Neisseria gonorrhoeae, Haemophilus influenzae, Shigela spp, Porphyromonas gingivalis, Fusobacterium nucleatum, Tannerella forsythensis, Treponema denticola, Aggregatibacter actinomycetemcomitans and Salmonella spp.

A peptide of the present document may be used alone or in combination with one or more other peptides of the present document.

A peptide based on SEQ ID NO: 1 with a carbon tail of 5, 6 or 7 carbons is a particularly preferred peptide in the context of the present document. Another particularly preferred peptide is SEQ ID NO: 3 with a carbon tail of 5, 6 or 7 carbons.

A peptide of the present document may for example be present in an amount of between 10 nM and 1 mM, for instance 1 μM. If two or more peptides are used together, the total concentration of peptides may be from 10 nM to 1 mM. Naturally, the amount of the peptide has to be adjusted to the specific composition and its intended use.

### Production of the Peptide(s)

Methods for synthesizing peptides are well-known to the skilled person and any method that results in a peptide according to the present document may be used.

A peptide of the present document is generally synthesized in vitro for example by solid phase peptide synthesis, by enzyme catalysed peptide synthesis or recombinant DNA technology.

The present document is therefore also directed to a nucleic acid sequence encoding a peptide as defined herein. Such a nucleic acid sequence may be inserted into a vector and expressed therefrom. For example, a cell, such as a bacterial cell, may be modified to produce a peptide as defined herein, e.g. by transforming said cell, such as a bacterial cell, with a vector or a nucleic acid sequence encoding a peptide as defined herein. The peptide may then be purified from said cell producing it, or the cell may be directly administered to the site where it is to act, e.g. to a wound. Alternatively, as mentioned elsewhere herein, the peptide may be produced as a pro-peptide, even when not explicitly mentioned.

### Compositions Comprising the Peptide(s)

A peptide as defined herein may be formulated as a pharmaceutical or a non-pharmaceutical composition, e.g. by formulating the peptide with common excipients, diluents, and/or carriers. Such excipients may e.g. be used to stabilize the peptide and suppress aggregation, and include, but is not limited to, solubilizers, surfactants, bulking agents (such as carbohydrates), thickeners (such as polymers) to increase solution viscosity, preservatives, vehicles, salts or sugars to stabilize peptides and to obtain physiological tonicity and osmolality and/or buffering agents to control pH.

A pharmaceutical or non-pharmaceutical composition according to the present document may in addition or alternatively comprise a nucleic acid sequence encoding a peptide of the present document, a vector comprising such a nucleic acid and/or a cell, for example a bacterial cell, able to express a peptide of the present document, e.g. by said cell comprising such a vector.

A pharmaceutical or a non-pharmaceutical composition according to the present document may comprise one or more of a peptide as defined herein, for example a peptide according to SEQ ID NO: 1 and/or SEQ ID NO: 2 and a peptide having an identity as specified herein to SEQ ID NO: 1 or SEQ ID NO: 2.

A pharmaceutical or a non-pharmaceutical composition comprising a peptide according to the present document may also comprise at least one further active compound, such as a drug, for example an antibiotic and/or an antimicrobial peptide not covered by the present document. An antibiotic may e.g. be selected from the group consisting of cell-wall inhibitors, DNA inhibitors, RNA inhibitors and protein inhibitors. Non-limiting examples of antibiotic that may be combined with a peptide of the present document are Vancomycin, Gentamicin, Tetracycline, Rifampicin, Ciprofloxacin and Cefotaxime.

The pharmaceutical or non-pharmaceutical composition may be formulated e.g. as a tablet, a capsule, a gel, a solution, a suspension, an emulsion, a powder, a cream, a paste, a lotion, a transdermal system, or an ointment.

Further, a peptide of the present document may be used in combination with a medical device. The present document therefore also relates to a medical device, such as a surgical suture, sheet, patch, membrane, hydrogel, coating, dressing and/or carrier material, comprising a peptide as defined herein, a nucleic acid sequence as defined herein, a vector as defined herein, a cell as defined herein and/or a pharmaceutical or non-pharmaceutical composition as defined herein. Such a medical device may provide antimicrobial, anti-inflammatory and/or antiparasitic properties to the medical device.

A peptide of the present document may for example be present in a pharmaceutical or a non-pharmaceutical composition in an amount of between 10 nM and 1 mM, for instance 1 μM. If two or more peptides are used together, the total concentration of peptides may be from 10 nM to 1 mM. Naturally, the amount of the peptide has to be adjusted to the specific composition and its intended use.

### Medical and Non-Medical Uses of the Peptide(s)

The peptide(s) disclosed herein have an activity, such as a biological activity, such as an antimicrobial, anti-inflammatory and/or antiparasitic activity comparable to the one of SEQ ID NO: 1 or SEQ ID NO: 2.

The present document therefore relates to a peptide and/or a pharmaceutical or non-pharmaceutical composition as defined herein for use in the treatment and/or prevention of a microbial and/or parasitic infection, an inflammation and/or a biofilm.

Likewise, the present document relates to a means being able to produce a peptide as defined herein, such as a nucleic acid sequence encoding such a peptide, a vector comprising such a nucleic acid sequence or a cell, such as a bacterial cell, expressing a peptide as defined herein, e.g. by comprising a vector as defined herein, for use in the treatment and/or prevention of a microbial and/or parasitic infection, an inflammation and/or a biofilm. When discussion the medical and non-medical uses of a peptide of the present document, whenever it is referred to “a peptide”, this also encompasses such means for producing a peptide of the present document, even if this is not explicitly mentioned.

The microbial infection may be a bacterial, a viral and/or a fungal infection. The bacteria may be either Gram-positive or Gram-negative bacteria. The bacterial infection may e.g. be generated by one or more bacteria selected from the group consisting of Staphylococcus spp (including MRSA, MRSE), Streptococcus spp (e.g. S. mutans, S. constellatus, S. anginosus), Enterococcus faecium (including VRE), Klebsiella pneumoniae, Acinetobacter baumannii, Pseudomonas aeruginosa, Enterobacter spp, Escherichia coli, Helicobacter pylori, Campylobacter, Salmonella, Neisseria gonorrhoeae, Haemophilus influenzae, Shigela spp, Porphyromonas gingivalis, Fusobacterium nucleatum, Tannerella forsythensis, Treponema denticola and Aggregatibacter actinomycetemcomitans and Salmonella spp.

The present document also relates to a peptide or a pharmaceutical composition according to the present document for use in disinfecting a wound and/or treating and/or preventing a microbial infection in a wound. In this case, a peptide or a pharmaceutical composition of the present document is administered topically or subcutaneously to the wound, e.g. in the form of a gel, a solution, a suspension, an emulsion, a powder, a cream, a paste, a lotion, a transdermal system, or an ointment. The wound may be any kind of wound, such as a blister wound, a soft tissue wound, a cutaneous wound, a deep wound, a cutaneous abscess, a surgical wound, a sutured laceration, a contaminated laceration, a burn wound, a decubitus ulcer, a statis ulcer, a leg ulcer, a foot ulcer, a venous ulcer, a diabetic ulcer, an ischemic ulcer, a pressure ulcer, an oral infection, a periodontal disease, a partial thickness burn and/or a full thickness burn.

Further, the present document relates to a peptide or a pharmaceutical or a non-pharmaceutical composition according to the present document for use in overcoming inherent or acquired resistance of a microorganism to an antibiotic.

A peptide or a pharmaceutical or a non-pharmaceutical composition according to the present document is administered in a manner suitable for the intended medical use of it. For example, a peptide or a pharmaceutical or a non-pharmaceutical composition according to the present document may be administered topically, orally, intravenously, subcutaneously and/or intramuscularly. The administration may be a single administration or the administration may be repeated one or more times at a suitable time interval, such as e.g. once a day, two times a day, or three times a day. The time period for the administration may be from one day to several days, such as 1 to 28 days or 1 to 14 days.

A peptide of the present document is administered in a pharmaceutically effective amount. This amount may differ depending on the actual condition to be treated and/or prevented.

A peptide of the present document may also be used to treat and/or prevent the formation of a biofilm. The biofilm may be a biofilm on a subject, such as a human, or it may be a biofilm formed on a non-living object.

A biofilm is a structured consortium of bacteria embedded in a self-produced polymer matrix consisting of polysaccharides, protein and extracellular DNA. Gradients of nutrients and oxygen exist from the top to the bottom of biofilms and the bacterial cells located in nutrient poor areas have decreased metabolic activity and increased doubling times. These more or less dormant cells are therefore responsible for some of the tolerance to antibiotics.

Bacterial biofilms are problematic as they may create resistance to antibiotics, disinfectant chemicals and to phagocytosis and other components of the innate and adaptive inflammatory defence system. As such, it is vital that a treatment can combat the formation of bacterial biofilms but also that it may disrupt an already existing biofilm.

Subjects to which a peptide of the present document may be administered include, but is not limited to, humans, mammals, birds and other vertebrate and non-vertebrate animals.

The present document is also directed to the use of a peptide as defined herein, a nucleic acid sequence as defined herein, a vector as defined herein, a cell as defined herein and/or a pharmaceutical or a non-pharmaceutical composition for the manufacture of a medicament for the treatment and/or prevention of a microbial and/or parasitic infection and/or inflammation. The microbial infection is generated by one or more bacteria selected from the group consisting of Staphylococcus spp, such as MRSA or MRSE, Streptococcus spp, such as S. mutans, S. constellatus or S. anginosus, Enterococcus faecium, such as VRE, Klebsiella pneumoniae, Acinetobacter baumannii, Pseudomonas aeruginosa, Enterobacter spp, Escherichia coli, Helicobacter pylori, Campylobacter, Salmonella, Neisseria gonorrhoeae, Haemophilus influenzae, Shigela spp, Porphyromonas gingivalis, Fusobacterium nucleatum, Tannerella forsythensis, Treponema denticola, Aggregatibacter actinomycetemcomitans and Salmonella spp.

The present document is also directed to the use of a peptide as defined herein, a nucleic acid sequence as defined herein, a vector as defined herein, a cell as defined herein, and/or a pharmaceutical or a non-pharmaceutical composition as defined herein for the manufacture of a medicament for disinfection a wound and/or treating and/or preventing a microbial infection in a wound, such as blister wound, a soft tissue wound, a cutaneous wound, a cutaneous abscess, a surgical wound, a sutured laceration, a contaminated laceration, a burn wound, a decubitus ulcer, a statis ulcer, a leg ulcer, a foot ulcer, a venous ulcer, a diabetic ulcer, an ischemic ulcer, a pressure ulcer, an oral infection, a periodontal disease, a partial thickness burn and/or a full thickness burn. The present document is also directed to the use of a peptide as defined herein, a nucleic acid sequence as defined herein, a vector as defined herein, a cell as defined herein, and/or a pharmaceutical composition as defined herein for the manufacture of a medicament for overcoming inherent or acquired resistance of a microorganism to an antibiotic.

The use of the peptide, nucleic acid sequence, vector, cell and/or pharmaceutical or a non-pharmaceutical composition as defined herein is administered topically, orally, intravenously, and/or intramuscularly.

The present document is also directed to a method for treating and/or preventing a microbial and/or parasitic infection and/or inflammation in a subject said method comprising administering to the subject in need thereof a pharmaceutically effective amount of a peptide as defined herein, a nucleic acid sequence as defined herein, a vector as defined herein, a cell as defined herein and/or a pharmaceutical or a non-pharmaceutical composition as defined herein.

The bacterial infection is generated by one or more bacteria selected from the group consisting of Staphylococcus spp, such as MRSA or MRSE, Streptococcus spp, such as S. mutans, S. constellatus or S. anginosus, Enterococcus faecium, such as VRE, Klebsiella pneumoniae, Acinetobacter baumannii, Pseudomonas aeruginosa, Enterobacter spp, Escherichia coli, Helicobacter pylori, Campylobacter, Salmonella, Neisseria gonorrhoeae, Haemophilus influenzae, Shigela spp, Porphyromonas gingivalis, Fusobacterium nucleatum, Tannerella forsythensis, Treponema denticola, Aggregatibacter actinomycetemcomitans and Salmonella spp.

The present document is also directed to method for disinfecting a wound, said method comprising administering an effective amount of a peptide as defined herein, a nucleic acid sequence as defined herein, a vector as defined herein, a cell as defined herein and/or a pharmaceutical or a non-pharmaceutical composition as defined herein to said wound, such as a blister wound, a soft tissue wound, a cutaneous wound, a cutaneous abscess, a surgical wound, a sutured laceration, a contaminated laceration, a burn wound, a decubitus ulcer, a statis ulcer, a leg ulcer, a foot ulcer, a venous ulcer, a diabetic ulcer, an ischemic ulcer, a pressure ulcer, an oral infection, a periodontal disease, a partial thickness burn and/or a full thickness burn.

The present document is also directed to method for overcoming inherent or acquired resistance of a microorganism to an antibiotic, said method comprising contacting said microorganism with peptide as defined herein, a nucleic acid sequence as defined herein, a vector as defined herein, a cell as defined herein and/or a pharmaceutical or a non-pharmaceutical composition as defined herein.

Said peptide, nucleic acid sequence, vector, cell and/or pharmaceutical composition is topically, orally, intravenously, and/or intramuscularly administered to said subject.

A peptide or a composition as defined herein may also be used as a non-medical antimicrobial, antiparasitic and/or disinfecting agent and/or for removing and/or preventing the formation of a biofilm on a non-biological object and/or as a biocide, optionally in combination with at least one further active compound, such as a drug, as defined elsewhere herein. The use as a biocide is preferably for cleaning and/or disinfecting non-biological objects.

A peptide of the present document may thus be used for cleaning and/or disinfecting non-biological objects, such as a non-biological surface. The peptide or a composition comprising said peptide is then spread over the surface to be treated, such as by spraying or wiping, and left to act. The peptide may then optionally be removed from the surface, e.g. by cleaning the surface with water. The peptide may thus be considered to be a disinfectant agent and/or a biocide. For example, a peptide of the present document or a composition comprising said peptide may be used for cleaning and/or disinfecting non-biological objects being contaminated with Gram-positive and/or Gram-negative bacteria, such as one or more bacteria selected from the group consisting of Staphylococcus spp (including MRSA, MRSE), Streptococcus spp (e.g. S. mutans, S. constellatus, S. anginosus), Enterococcus faecium (including VRE), Klebsiella pneumoniae, Acinetobacter baumannii, Pseudomonas aeruginosa, Enterobacter spp, Escherichia coli, Helicobacter pylori, Campylobacter, Salmonella, Neisseria gonorrhoeae, Haemophilus influenzae, Shigela spp, Porphyromonas gingivalis, Fusobacterium nucleatum, Tannerella forsythensis, Treponema denticola and Aggregatibacter actinomycetemcomitans and Salmonella spp.

The peptide or composition comprising said peptide, as defined elsewhere herein, may comprise said peptide in a concentration range selected from 120 μM to 2.5 μM, 100 μM to 3 μM, 80 μM to 5 μM, 60 μM to 10 μMor 40 μM to 15 μM. The peptide or composition comprising said peptide may be administered at least one time to a subject, such as at least 2 times, at least 3 times, at least 4 times or at least 5 times.

The invention will be further described in the following examples, which do not limit the scope of the invention described in the claims.

### EXPERIMENTAL SECTION

### Material and Methods

### Bacterial Culture

S. aureus (ATCC 29213, MSSA, ATCC, Manassas, VA) and E. coli (K-12 MG1655) were streaked on Luria-Bertani (LB) agar plates and incubated at 37° C. overnight. Single colonies were inoculated into 5 ml of LB broth and incubated on a shaker (400 rpm) at 37° C. overnight. The bacterial concentrations were determined by viable count and were adjusted to correlate with approximately 10⁹ CFU/ml.

### Peptide Design and Prediction of Antimicrobial Activity

PLNC8 β sequence of the bacteriocin PLNC8 αβ was used to design new and optimized antimicrobial peptides. The sequence of PLNC8 β was truncated to 16 amino acids (residue #1-16), after which one amino acid was removed from the N-terminal region and one amino acid added to the C-terminal region. This was completed throughout the entire sequence of PLNC8 β, which is composed of 34 amino acids. The antimicrobial activity of full length and truncated peptides of PLNC8 β was predicted using the servers AntiBPs (* https://webs.iiitd.edu.in/raghava/antibp2/submit.html) and ADAM (#http://bioinformatics.cs.ntou.edu.tw/adam/svm predict.php). The sequence with the highest score of predicted antimicrobial activity was used to generate new variants. Peptide characteristics, such as molecular weight, chemical formula, iso-electric point, net charge at pH 7, and percentage of hydrophobic, acidic, basic and neutral residues were determined using Peptide 2.0 (https://www.peptide2.com/). Peptide structures were predicted using the PEP-FOLD tool in the RPBS Web Portal (https://bioserv.rpbs.univ-paris-diderot.fr/index.html).

All the sequences showing characteristics of being antimicrobial peptides were synthesized and tested against gram positive (S. aureus) and gram negative (E. coli) bacteria.

Sequences referred to in the present document can be found in the appended sequence listing and in the below Table 5 where they are identified by their respective SEQ ID Nos.

### Peptide Synthesis

The peptides were synthesized using Fmoc-chemistry on an automated microwave peptide synthesizer (CEM) in 25-100 μM scale. ProTide Rinkamide resin was used as solid support for all synthesis yielding an amidated C-terminal. Fmoc-protected amino acids were sequentially coupled using a five-fold excess of amino acid, Oxyma as base and DIC as coupling reagent in DMF under microwave conditions. Fmoc-deprotection was achieved by treatment with 20% Piperidine in DMF under microwave conditions. After final Fmoc-deprotection N-terminal modification was achieved by treatment of the resin-bound peptide with Acetic anhydride in DMF (1:1) for acetylation, or n-Alkane acids (10 eq) combined with HCTU (10 eq) and DIPEA (20 eq) in DMF for lipidation. Global deprotection and cleavage of peptides from resin was achieved by treatment with TFA:TIS:H₂O (95/2.5/2.5, v/v/v) for 3 hours before being concentrated using a stream of nitrogen. The crude peptides were precipitate in ice cold diethyl ether, twice, and the ether was discarded. The crude peptides were purified on a semi preparative HPLC system (Dionex) equipped with a RP C-18 column (ReproSil Gold) using an aqueous gradient of acetonitrile containing 0.1% TFA. Purity was controlled using an analytical column (C-18, Supelcosil) attached to the same HPLC system and peptide identity was confirmed using Maldi-ToF mass spectrometer (Bruker).

### Antimicrobial Activity

The broth microdilution method was used to determine minimal inhibitory concentration (MIC) and minimal bactericidal concentration (MBC). Two-fold serial dilutions of the peptides were used, and the final concentrations ranged from 0.19-100 μM. The effect of peptide-antibiotic combinations was investigated by performing a checkerboard analysis. The final concentrations of the antibiotics vancomycin, gentamicin and rifampicin ranged between 0.39 and 50 μg/ml, while levofloxacin ranged between 0.031 and 4 μg/ml (S. aureus). The MIC-values were determined visually and spectroscopically (620 nm) as the first concentration that completely inhibited bacterial growth. All concentrations that resulted in complete inhibition of bacterial growth were cultured (10 μl) on LB-agar plates, and the lowest concentration where no growth was observed represented the MBC. The fractional inhibitory concentration (FIC) and fractional bactericidal concentration (FBC) were calculated by the equation (MIC or MBC of peptide in combination/MIC or MBC of peptide αβ alone)+(MIC or MBC of antibiotic in combination/MIC or MBC of antibiotic alone). Synergy was defined as FIC/FBC≤0.5, additive when 0.5<FIC/FBC≤1, indifferent when 1<FIC/FBC<2 and antagonistic when FIC/FBC≥2. All experiments were repeated at least three times.

### Hemolytic Activity

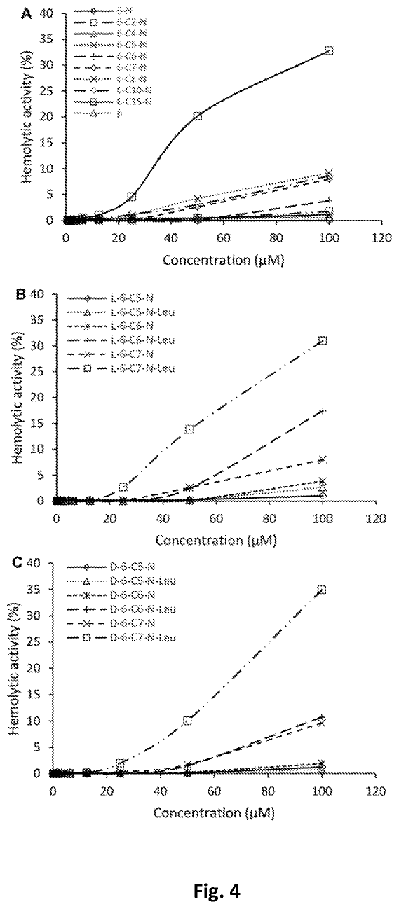

The hemolytic activity of the peptides was investigated by collecting blood from healthy volunteers in heparinized vacutainers. Briefly, the blood was centrifuged at 600×g for 5 min and the erythrocyte pellet was washed three times in PBS. The cells were then suspended in PBS and added to 96-well plates (15% erythrocyte suspension/well), containing the peptides with two-fold serial dilution. The plates were incubated for 1 h at 37° C. followed by centrifugation for 5 min at 900×g and absorbance measurement of the supernatants at 540 nm. Hemolytic activity (%) was calculated by subtracting the negative control from all values and normalization against the positive control (0.5% Triton X-100), that was set to 100%. All experiments, each in duplicate, were repeated three times. The results are presented in FIG. 4.

### Microscopy

The fluorescent dye Sytox® Green was used to investigate membrane permeabilization caused by PLNC8 αβ. This fluorophore can only cross damaged membranes and fluoresce upon binding to nucleic acids. The bacteria were washed and resuspended in PBS and incubated for 5 min with or without peptides in 96-well microtiter plates. Images were captured with Olympus BX41 and the fluorescence intensity was analysed and quantified using the software ImageJ.

### Ethics Statement

Ethical permission for collecting heparinized blood from healthy volunteers was approved by the regional ethical board at Orebro-Uppsala County (Dnr 2015/543). Informed consent was obtained from all volunteers. Collection of blood and associated methods were carried out in accordance with relevant guidelines and regulations.

### Results

It has been shown that the antimicrobial activity of the bacteriocin PLNC8 αβ against Staphylococcus spp. is optimal when both the α and β peptides are used together in a molar ratio of 1:1¹⁴. PLNC8 αβ is an efficient and valuable bacteriocin against gram-positive bacteria, however it would be advantageous to develop short and optimized peptides of PLNC8 αβ with broad spectrum activity to also target challenging infections caused by gram-negative bacteria. PLNC8 β, but not PLNC8 α, showed activity on liposomes and against bacteria, but it was not sufficient to inhibit bacterial growth.

Development of short and optimized antimicrobial peptides was therefore based on the amino acid sequence of PLNC8 β.

Identification of short antimicrobial peptides was initiated by generating a small library of truncated sequences of PLNC8 β (34 residues), each composed of 16 amino acid residues, to contain at least 3-4 helix turns, and resulted in 19 peptides (Table 1). Ten of these truncated variants were predicted (AntiBPs and ADAM servers) to be antimicrobial peptides. Full-length PLNC8 β was not predicted to have antimicrobial activity compared to the human cathelicidin-derived peptide LL-37. The sequence with the highest scores in both servers was further used to generate new modified peptides, which was achieved by considering e.g. their amphipathic interfaces through projection of their respective helical wheel. Peptides with a strict amphipathic conformation are avoided since they are known to be highly cytotoxic and cause instant cell death through membrane permeabilization¹⁸.

TABLE 1
Prediction of the antimicrobial activities of truncated and modified
peptides of PLNC β-The antimicrobial activity of full length and truncated
peptides of PLNC8 13 was predicted using the servers AntiBPs (*
https://webs.iiitd. edu. in/raghava/antibp2/submit.html) and ADAM(#
http://bioinformatics.cs.ntou.edu.tw/adam/svm_predict.php).
Peptides that were predicted to have antimicrobial
activity are highlighted in boxes and the variants
with the highest score, highlighted in gray, were
synthesized for analysis in biological systems.

| Sequence | Name | Score* | Score# |
|---|---|---|---|
| SVPTSVYTLGIKILWSAYKHRKTIEKSFNKGFYH | PLNC8 β | −0.24 | −0.44 |
| SVPTSVYTLGIKILWS | | | |
| VPTSVYTLGIKILWSA | | | |
| PTSVYTLGIKILWSAY | | | |
| TSVYTLGIKILWSAYK | 1 | 0.132 | 0.67 |
| SVYTLGIKILWSAYKH | 2 | 0.295 | 0.9 |
| VYTLGIKILWSAYKHR | 3 | 0.016 | 0.52 |
| YTLGIKILWSAYKHRK | 4 | 0.558 | 0.95 |
| TLGIKILWSAYKHRKT | 5 | 0.435 | 1.16 |
| LGIQILWSAYKHRKTI | 6 | 0.783 | 1.1 |
| GIKILWSAYKHARKTIE | 7 | 0.296 | 0.95 |
| IKILWSAYKHRKTIEK | 8 | 0.031 | 0.68 |
| KILWSAYKHRKTIEKS | | | |
| ILWSAYKHRKTIEKSF | | | |
| LWSAYKHRKTIEKSFN | | | |
| WSAYKHRKTIEKSFNK | | | |
| SAYKHRKTIEKSFNKG | | | |
| AYKHRKTIEKSFNKGF | 9 | 0.154 | 0.35 |
| YKHRKTIEKSFNKGFY | | | |
| KHRKTIEKSFNKGFYH | 10 | 0.003 | 0.64 |
| LGIKILRSAYKHRKTIEK | 11 | 0.606 | 0.39 |
| LGIKILRSAYKHRKTIEKSFNK | 12 | 0.447 | −0.02 |
| LGQKILRSARKFGKTIEKSF | 13 | 1.222 | 1.12 |
| LGQKILRSARKFGKDIEKSF | 14 | 1.438 | 1.31 |
| LGIKILRSARKFGKVIEKSF | 15 | 1.596 | 1.53 |
| LGIKILWSARKFGKVIEKSF | 16 | 1.506 | 1.79 |
| LLGIKILWSARKFGKVIEKSF | 17 | 1.735 | 1.73 |
| LLGIKILWKARKFGKVIEKSF | 18 | 1.858 | 2.11 |
| KILWSAYKHR | S1 | 1.37 | |
| ILWSAYKHR | S2 | 1.36 | |
| LWSAYKHR | S3 | 1.27 | |
| WSAYKHR | S4 | 1.8 | |
| WSAYKH | S5 | 2.3 | |
| WSAYK | S6 | 2.19 | |
| LLGDFFRKSKEKIGKEFKRIVQRIKDFLRNLVPRTES | LL-37 | 1.474 | 1.06 |

Full length PLNC8 β (first sequence, 34 amino acids) was predicted to not be an antimicrobial peptide compared to LL-37.

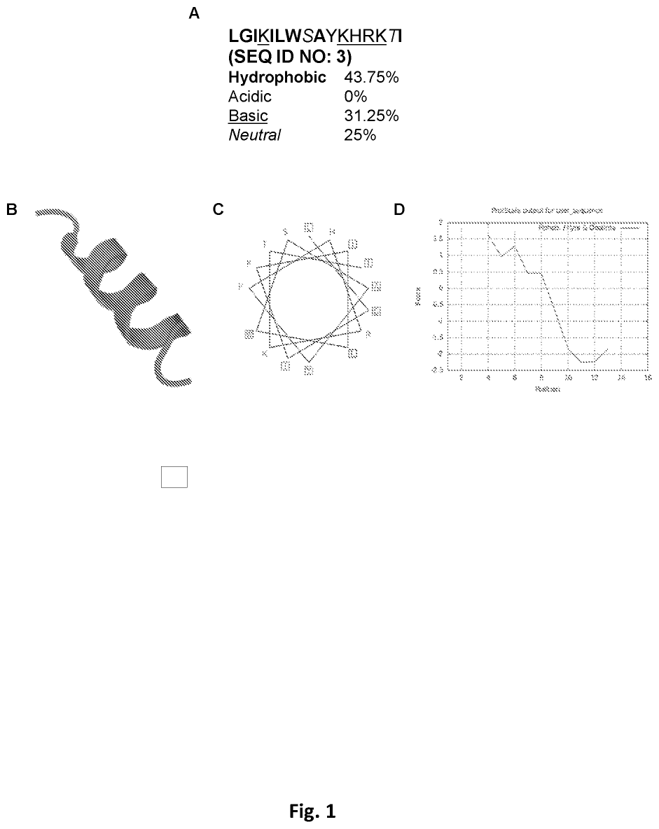

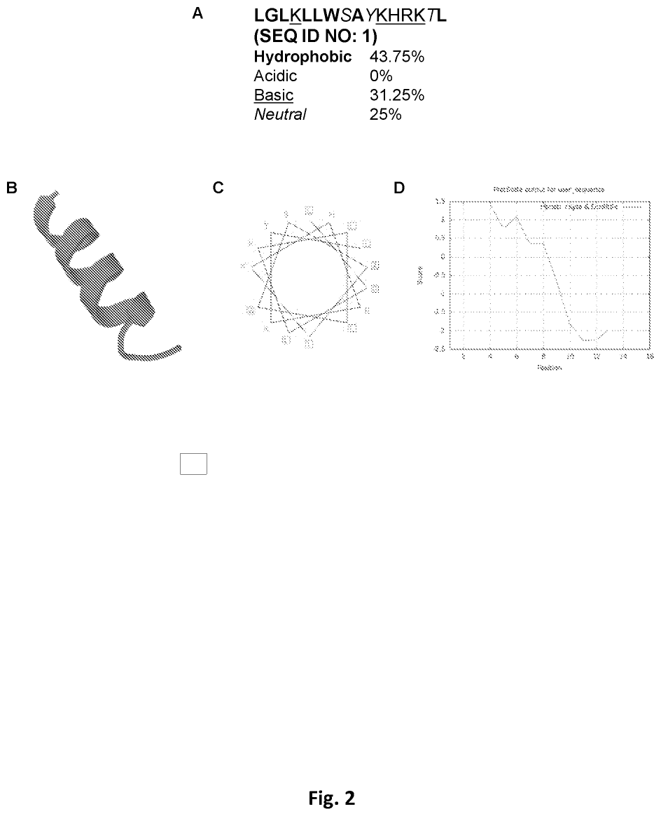

All peptides were synthesized with an acetylated N-terminal region and amidated C-terminal region. These post-translational modifications of proteins and peptides are common and important in eukaryotic cells by increasing stability and preventing degradation. The antimicrobial activities of the new peptides were determined on S. aureus and E. coli and revealed a short peptide of 16 amino acid (#6, SEQ ID NO: 3)) with inhibitory and bactericidal activity against both bacteria at 100 UM and 50 μM, respectively (Table 2). The antimicrobial activity of peptide 6 (SEQ ID NO: 3) against E. coli was comparable to the activity of LL-37, while full-length PLNC8 β showed no activity. The characteristics of peptide #6 (SEQ ID NO: 3) are shown in FIG. 1, including the distribution of amino acid residues (FIG. 1A), peptide structure as determined in silica using PEP-FOLD, which has an Optimized Potential for Efficient Structure Prediction (sOPEP) value of −28.2612 (FIG. 1B), helical wheel projection (FIG. 1C), and hydrophilicity plot (FIG. 1D). The probability that this peptide would be toxic to human cells is small since the arrangement of amino acids indicate lack of strict amphipathicity. The corresponding data for a peptide according to SEQ ID NO: 1 is presented in FIG. 2, which has an Optimized Potential for Efficient Structure Prediction (sOPEP) value of −32.3544 (FIG. 2B).

TABLE 2
Antimicrobial activity of full length and truncated peptides of PLNC β-
The antimicrobial activity was determined against gram-positive S. aureus
and gram-negative E. coli using the broth microdilution method. The
truncated peptide #6 (SEQ ID NO: 3), which is composed of 16 amino acids,
but not full length PLNC8 β, was found to possess inhibitory and
bactericidal activity against both S. aureus and E. coli.
The human-derived antimicrobial peptide LL-37 was used as a control.

| | | | S. aureus | | E. Coli | |
|---|---|---|---|---|---|---|
| Sequence | Name | MW | MIC | MBC | MIC | MBC |
| [symbol] | β | [symbol] | >100 | >100 | >100 | >100 |
| [symbol] | 4 | [symbol] | >100 | >100 | >100 | >100 |
| [symbol] | 5 | [symbol] | >100 | >100 | >100 | >100 |
| [symbol] | 6 | [symbol] | 100 | 100 | 50 | 50 |
| [symbol] | 13 | [symbol] | >100 | >100 | >100 | >100 |
| [symbol] | 14 | [symbol] | >100 | >100 | >100 | >100 |
| [symbol] | 15 | [symbol] | >100 | >100 | >100 | >100 |
| [symbol] | 16 | [symbol] | >100 | >100 | >100 | >100 |
| [symbol] | 17 | [symbol] | >100 | >100 | >100 | >100 |
| [symbol] | 18 | [symbol] | >100 | >100 | 100 | >100 |
| [symbol] | S1 | [symbol] | >100 | >100 | >100 | >100 |
| [symbol] | S2 | [symbol] | >100 | >100 | >100 | >100 |
| [symbol] | S3 | [symbol] | >100 | >100 | >100 | >100 |
| [symbol] | S4 | [symbol] | >100 | >100 | >100 | >100 |
| [symbol] | S5 | [symbol] | >100 | >100 | >100 | >100 |
| [symbol] | S6 | [symbol] | >100 | >100 | >100 | >100 |
| [symbol] | LL-37 | [symbol] | >100 | >100 | 50 | 50 |

[symbol]

indicates data missing or illegible when filed

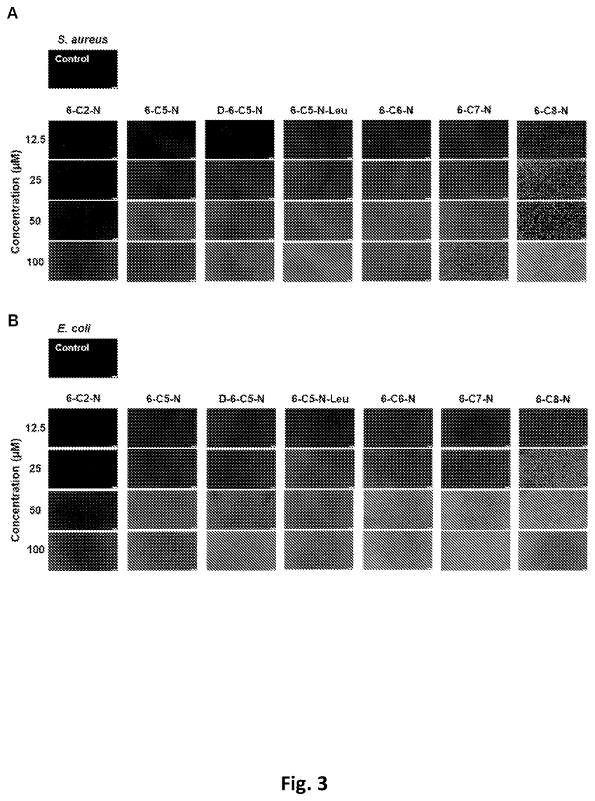

SEQ ID NO 3 (Peptide #6, SEQ ID NO: 3) was selected as the lead compound for additional modifications to improve its stability and antimicrobial activity upon interaction with bacterial membranes, including fatty acid conjugation and PEGylation of the N—S terminal region. PEGylation did not enhance the antimicrobial activity of peptide #6 (SEQ ID NO: 3), while fatty acid conjugation markedly increased the activity in a length-dependent manner (Table 3), reaching its maximum at five to seven carbon atoms (6-C5-N, 6-C6-N, and 6-C7-N). Although elongation of the fatty acid chain to eight carbon atoms (6-C8-N) did not affect the antimicrobial activity of the lipopeptide against S. aureus, it certainly affected the activity against E. coli as the MIC and MBC values increased from 6.2 μM to 12.5 μM, respectively. Conjugation of the peptide with a fatty acid chain of ≥10 carbon atoms drastically suppressed bacterial inhibition and completely abolished the bactericidal effects. Chu-Kung and colleagues 19 showed an increased antimicrobial activity of peptides following conjugation of lauric acid (fatty acid chain with a 12 carbon atom backbone). Obtaining a significantly improved antimicrobial activity after conjugation of short fatty acid chains (five to seven carbon atoms) is thus surprising. Furthermore, the D-enantiomers of 6-C5-N, 6-C6-N, and 6-C7-N showed enhanced antimicrobial activity against both S. aureus and E. coli, which is probably due to resistance against proteolytic degradation. These results indicate that the peptide does not recognize and bind to specific target molecules, e.g., proteins and glycoproteins, on the bacterial surface, suggesting that the initial binding is driven by electrostatic interactions with anionic bacterial structures, such as membrane lipids. An additional modification of the lipopeptides 6-C5-N, 6-C6-N, and 6-C7-N included replacement of all three isoleucine residues with leucine, which surprisingly improved their antimicrobial activity against S. aureus and led to SEQ ID NO: 1. Fluorescence microscopy showed that the antimicrobial effect of the peptides is rapid, achieving substantial bacterial lysis already after 5 min in a dose-dependent manner (FIG. 3), which was associated with the length of the fatty acid chain. In other words, Conjugation of the peptide with a fatty acid chain of five to eight carbon atoms was efficient at quickly permeabilizing both gram-positive and gram-negative bacterial membranes. Although the precise mechanisms remain to be determined, including initial interactions with the bacterial cell wall (gram-positive) or outer membrane (gram-negative), the rapid permeabilization indicates that the final and main target of the lipopeptides is bacterial membranes.

TABLE 3
Antimicrobial activity of modified variants of peptide sequence #6
(SEQ ID NO: 3). The amino-terminal region of the truncated peptide was either left
unmodified, acetylated or pegylated. The antimicrobial activities were determined
against S. aureus and E. coli. Acetylation with five carbon atoms substantially
enhanced the inhibitory and bactericidal activity of the truncated peptide against both
bacteria. Full length PLNC8 B was used for comparison and as a control. The D-
enantiomer of 6-C5-10 N showed an enhanced inhibitory and bactericidal activity.
Interestingly, replacement of all three isoleucine residues with leucine (resulting in
SEQ ID NO: 1) in 6-C5-N improved the antimicrobial activity against S. aureus.

| | | | S. aureus | | E. Coli | |
|---|---|---|---|---|---|---|
| Sequence | Name | MW | MIC | MBC | MIC | MBC |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |

[symbol]

indicates data missing or illegible when filed

Cytotoxicity and hemolytic activity of synthetic antimicrobial peptides has been problematic and is an important aspect to carefully evaluate. Hemolytic activity was found to be associated with the length of the fatty acid chain, where ≤6 carbon atoms caused less than 5% hemolysis at 100 μM, 7-10 carbon atoms resulted in around 10% hemolysis, and a fatty acid chain of 15 carbon atoms caused about 40% hemolysis (FIG. 4A). the L-enantiomers of 6-C5-N, 6-C6-N, and 6-C7-N showed increased hemolytic activity, reaching ˜10% with C7, and their corresponding leucine variants increased the hemolytic activity further (FIG. 4B). The D-enantiomers of 6-C5-N, 6-C6-N, and 6-C7-N showed similar hemolytic activity as the L-forms (FIG. 4C). Laverty et al., 2010²⁰ analyzed a range of lipopeptide with different lengths of fatty acids, and showed that the antimicrobial activity was enhanced by increasing the length of the fatty acid chain. The most effective variant comprised a fatty acid chain with 12 carbon atoms, which is in line with the results obtained by Chu-Kung and colleagues¹⁹.

However, this lipopeptide was shown to be highly hemolytic and cytotoxic at concentrations ≥50 μg/ml, resulting in complete hemolysis.

Antimicrobial peptides that target bacterial membranes are attractive candidates to be used in combination with antibiotics to increase the efficacy, reduce the overall use of antibiotics, and consequently suppress the development and spreading of antimicrobial resistance. The lipopeptides 6-C2-N and 6-C5-N were combined with different antibiotics and the antimicrobial activity was determined against E. coli and S. aureus. Both lipopeptides were able to decrease the concentrations of antibiotics required to inhibit and kill the bacteria in an additive or synergistic manner (Table 4). The lipopeptides may thus be used in combination therapy to reduce the concentration of antibiotics, and consequently reduce possible side-effects, environmental contaminations, and development of resistance. Nisin has been shown to act synergistically with citric acid²¹, penicillin, and chloramphenicol²² against several Staphylococcus species. It has previously been shown that plantaricin A, E, F, J, K²³, and NC8 αβ¹⁴ substantially enhances the effects of several antibiotics, including vancomycin, teicoplanin, rifampicin, gentamicin, and tetracycline, against Staphylococcus species.

TABLE 4
Antimicrobial activity of acetylated peptides in combination with
antibiotics. The antimicrobial activities of acetylated peptides with two and five
carbon atoms, with or without different antibiotics, were determined in a checkerboard
analysis against A) S. aureus and B) E. coli. The fractional inhibitory concentration
(FIC) and fractional bactericidal concentration (FBC) shows that the peptides interact
with the antibiotics in a synergistic or additive manner. Synergy was defined as
FIC/FBC ≤ 0.5, additive when 0.5 < FIC/FBC ≤ 1, indifferent when 1 < FIC/FBC < 2
and antagonistic when FIC/FBC ≥ 2.

| A | S. aureus | | |
|---|---|---|---|
| Antimicrobial agent | MIC | MBC | [symbol] FIC/FBC |
| [symbol] | 100 | 100 | |
| [symbol] | 6.25 | 6.25 | |
| [symbol] | 0.78 | 0.78 | |
| [symbol] | [symbol] | [symbol] | [symbol] |
| [symbol] | [symbol] | [symbol] | [symbol] |
| [symbol] | 0.62 | 0.62 | 0.75/1.00 |
| [symbol] | [symbol] | [symbol] | [symbol] |
| [symbol] | [symbol] | [symbol] | [symbol] |
| [symbol] | 0.25 | 0.25 | [symbol] |
| [symbol] | [symbol] | [symbol] | [symbol] |
| [symbol] | [symbol] | [symbol] | [symbol] |
| B | E.Coli | | |
| Antimicrobial agent | MIC | MBC | [symbol] FIC/FBC |
| [symbol] | 50 | 50 | |
| [symbol] | 12.5 | 12.5 | |
| Gentamicin(μl) | [symbol] | [symbol] | |
| [symbol] | [symbol] | [symbol] | [symbol] |
| [symbol] | [symbol] | [symbol] | [symbol] |
| [symbol] | 25 | 100 | |
| [symbol] | [symbol] | [symbol] | [symbol] |
| [symbol] | [symbol] | [symbol] | [symbol] |

[symbol]

indicates data missing or illegible when filed

TABLE 5
Listing of peptide sequences referred to
in the present document

| Amino acid sequence | Example | TABLE | SEQ ID NO |
|---|---|---|---|
| LGLKLLWSAYKHRKTL | | 3 | 1 |
| LTKRHKYASWLLKLGL | | | 2 |
| LGIKILWSAYKHRKTI | | 1; 2; | 3 |
| | | 3; 4 | |
| ITKRHKYASWLIKIGL | | | 4 |
| SVPTSVYTLGIKILWS | PLNC8 β | 1; 2; | 5 |
| AYKHRKTIEKSFNKGFYH | | 3 | |
| SVPTSVYTLGIKILWS | | 1 | 6 |
| VPTSVYTLGIKILWSA | | 1 | 7 |
| PTSVYTLGIKILWSAY | | 1 | 8 |
| TSVYTLGIKILWSAYK | | 1 | 9 |
| SVYTLGIKILWSAYKH | | 1 | 10 |
| VYTLGIKILWSAYKHR | | 1 | 11 |
| YTLGIKILWSAYKHRK | | 1; 2 | 12 |
| TLGIKILWSAYKHRKT | | 1; 2 | 13 |
| GIKILWSAYKHRKTIE | | 1 | 14 |
| IKILWSAYKHRKTIEK | | 1 | 15 |
| KILWSAYKHRKTIEKS | | 1 | 16 |
| ILWSAYKHRKTIEKSF | | 1 | 17 |
| LWSAYKHRKTIEKSFN | | 1 | 18 |
| WSAYKHRKTIEKSFNK | | 1 | 19 |
| SAYKHRKTIEKSFNKG | | 1 | 20 |
| AYKHRKTIEKSFNKGF | | 1 | \|21 |
| YKHRKTIEKSFNKGFY | | 1 | \|22 |
| KHRKTIEKSFNKGFYH | | 1 | 23 |
| LGIKILRSAYKHRKTIEK | | 1 | 24 |
| LGIKILRSAYKHRKTIEKSFNK | | 1 | 25 |
| LGQKILRSARKFGKTIEKSF | | 1; 2 | 26 |
| LGQKILRSARKFGKDIEKSF | | 1; 2 | 27 |
| LGIKILRSARKFGKVIEKSF | | 1; 2 | 28 |
| LGIKILWSARKFGKVIEKSF | | 1; 2 | 29 |
| LLGIKILWSARKFGKVIEKSF | | 1; 2 | 30 |
| LLGIKILWKARKFGKVIEKSF | | 1; 2 | 31 |
| KILWSAYKHR | | 1; 2 | 32 |
| ILWSAYKHR | | 1; 2 | 33 |
| LWSAYKHR | | 1; 2 | 34 |
| WSAYKHR | | 1; 2 | 35 |
| WSAYKH | | 1; 2 | 36 |
| WSAYK | | 1; 2 | 37 |
| LLGDFFRKSKEKIGKEFKRI | LL-37 | 1; 2 | 38 |
| VQRIKDFLRNLVPRTES | | | |

It was evaluated whether the peptide SEQ ID NO:3 having L-amino acids and an acetylation with five carbon atoms at the N-terminal portion (L-6-C5-N) could effectively reduce an inflammatory response to a bacterial infection to HaCaT keratinocytes. After 24 hours of exposure to Staphylococcus aureus (MOI=0.1), and three administrations of L-6-C5-N SEQ ID NO:3 in different concentrations (2.5, 5, 10, 20 and 40 μM) at 1, 5 and 9 hours post infection, a LDH cytotoxicity assay was performed and IL-6, TNF-α and CXCL-8 concentrations were measured. The symbol (−) indicates negative control (uninfected/unstimulated cells), (+) indicates positive control (S. aureus-infected cells).

Statistical analysis was done by ANOVA with Tukey's post hoc test. P-values are indicated by *=0.5, ***=0.001.

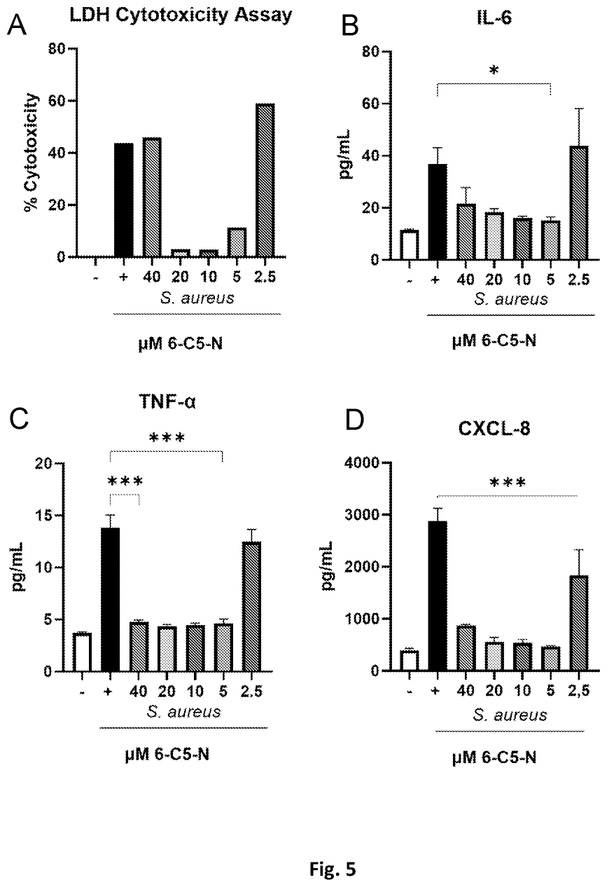

As shown in FIG. 5A, cell lysis of HaCaT keratinocytes infected with S. aureus was significantly reduced after administration of L-6-C5-N SEQ ID NO:3, particularly in a concentration higher than 2.5 μm and lower than 40 μm.

Furthermore, pro-inflammatory cytokines IL-6, TNF-α and CXCL-8 concentrations have been measured after administration of L-6-C5-N SEQ ID NO:3 as described above. IL-6 secretion was significantly reduced when compared with the positive control (infected cells without addition of L-6-C5-N SEQ ID NO:3). Said reduction was more significant from 5 to 40 μM of L-6-C5-N SEQ ID NO:3 (FIG. 5B). Similar results have been found when measuring TNF-α (FIG. 5C) and CXCL-8 (FIG. 5D), which indicates that L-6-C5-N SEQ ID NO:3 was effective in reducing or eliminating S. aureus, and thereby reducing an inflammatory response. According to these results, an effective amount L-6-C5-N SEQ ID NO:3 is higher than 2.5 μm and lower than 40 μm.

FIG. 5 surprisingly shows that even low doses of L-6-C5-N SEQ ID NO:3 are very effective in reducing or eliminating bacterial infections.

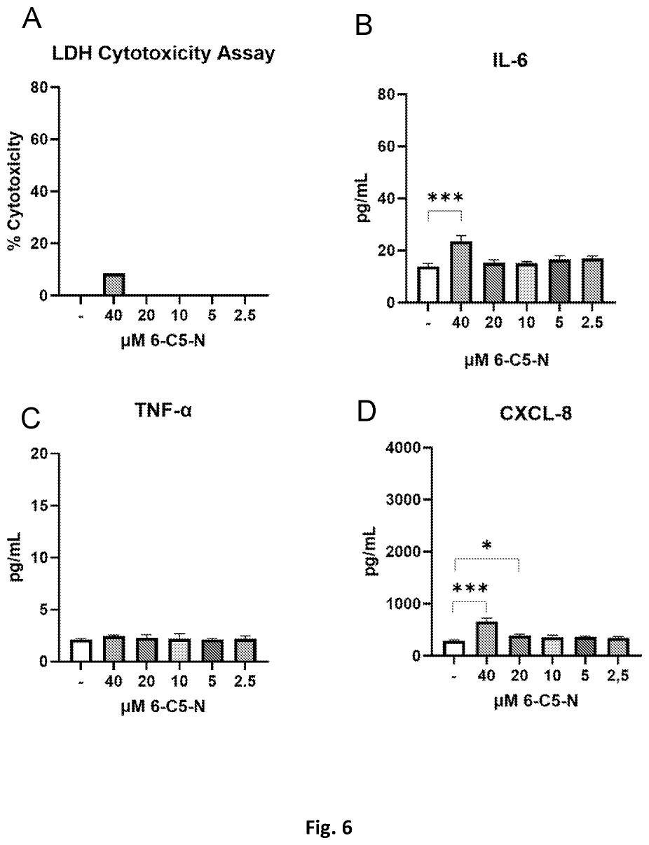

To evaluate whether L-6-C5-N SEQ ID NO:3 can generate an undesired inflammatory response and/or keratinocyte cell lysis, the experiment was repeated in uninfected HaCaT keratinocytes (FIG. 6). After 24 hours of exposure to three administrations of L-6-C5-N SEQ ID NO:3 in different concentrations (2.5, 5, 10, 20 and 40 μM) at 0, 4 and 8 hours, a LDH cytotoxicity assay was performed and IL-6, TNF-α and CXCL-8 concentrations were measured. The symbol (−) indicates negative control. Statistical analysis was done by ANOVA with Tukey's post hoc test. P-values are indicated by *=0.5, ***=0.001.

FIG. 6A surprisingly shows that even high doses of L-6-C5-N SEQ ID NO:3 do not significantly promote keratinocyte cell lysis. When measuring cytokines IL-6, TNF-α and CXCL-8 after L-6-C5-N SEQ ID NO:3 administration, it was found that no or almost no cytokine secretion is detected, which indicates that the peptide does not significantly stimulate an inflammatory response (FIGS. 6B, 6C and 6D).

Said results clearly indicate that the peptides according to the disclosure advantageously provide an effective antimicrobial response that simultaneously does not significantly promote human cell lysis and/or inflammation.

It is to be understood that while the invention has been described in conjunction with the detailed description thereof, the foregoing description is intended to illustrate and not limit the scope of the invention, which is defined by the scope of the appended claims.

Other aspects, advantages, and modifications are within the scope of the following claims.

Unless expressly described to the contrary, each of the preferred features described herein can be used in combination with any and all of the other herein described preferred features.

We claim:

1. An isolated peptide or a salt thereof, such as a pharmaceutically acceptable salt thereof, said peptide:
(a) comprising an amino acid sequence as set forth in SEQ ID NO: 1 or SEQ ID NO: 2 or an amino acid sequence having at least 85% or at least 90%, sequence identity to SEQ ID NO: 1 or SEQ ID NO: 2; or
(b) consisting of an amino acid sequence as set forth in SEQ ID NO: 1 or SEQ ID NO: 2 or an amino acid sequence having at least 75%, such as at least 80%, at least 85%, or at least 90%, sequence identity to SEQ ID NO: 1 or SEQ ID NO: 2.

2. The peptide according to claim 1, wherein the peptide is selected from the group consisting of SEQ ID NO: 1, SEQ ID NO: 2, SEQ ID NO: 3 and SEQ ID NO: 4.

3. The peptide according claim 1, wherein at least one amino acid residue is a D amino acid, preferably all amino acid residues being D amino acids.

4. The peptide according to claim 1, wherein at least one amino acid residue is a L amino acid, preferably all amino acid residues being L amino acids.

5. The peptide according to claim 1, wherein the peptide further comprises a hydrophobic moiety bound to the peptide, such as an acyl group, a hydrophobic amino acid, or a lipid covalently bound to the peptide.

6. The peptide according to claim 5, wherein the hydrophobic moiety is covalently bound to a N-terminal end of the peptide.

7. The peptide according to claim 5, wherein the hydrophobic moiety is an acyl group comprising a carbon chain length of at least 1 carbon.

8. The peptide according to any one of claim 5, wherein the hydrophobic moiety is an acyl group selected from the group consisting of acetyl, propionyl, butyryl, pentanoyl, hexanoyl, heptanoyl, octanoyl and decanoyl, preferably pentanoyl, hexanoyl, heptanoyl.

9. The peptide according to claim 1, wherein the amino acid sequence of said peptide in one or more positions comprises one or more of a substitution, deletion and/or insertion and/or conservative amino acid substitutions and/or conservatively modified sequence variants.

10. The peptide according to claim 1, wherein the peptide has an antimicrobial, antiparasitic and/or anti-inflammatory activity.

11. The peptide according to claim 10, wherein said antimicrobial activity is an antibacterial, antiviral and/or antifungal activity.

12. The peptide according to claim 1, wherein the peptide has an antimicrobial activity against one or more gram-negative and/or gram-positive bacteria.

13. The peptide according to claim 1, wherein the peptide has an antimicrobial activity against one or more bacteria selected from the group consisting of Staphylococcus spp, such as MRSA or MRSE, Streptococcus spp, such as S. mutans, S. constellatus, S. anginosus, Enterococcus faecium, such as VRE, Klebsiella pneumoniae, Acinetobacter baumannii, Pseudomonas aeruginosa, Enterobacter spp, Escherichia coli, Helicobacter pylori, Campylobacter, Salmonella, Neisseria gonorrhoeae, Haemophilus influenzae, Shigela spp, Porphyromonas gingivalis, Fusobacterium nucleatum, Tannerella forsythensis, Treponema denticola und Aggregatibacter actinomycetem-comitans and Salmonella spp.

14. The peptide according to claim 1, wherein said peptide has a length of from 12 to 100 amino acids.

15. The peptide according to claim 1, wherein said peptide is in the form of a pro-peptide and said peptide is produced by degradation of said pro-peptide.

16. The peptide according to claim 1, wherein said peptide is in a concentration range selected from 120 μM to 2.5 μM, 100 μM to 3 μM, 80 μM to 5 μM, 60 μM to 10 μM or 40 μM to 15 μM.

17.-41. (canceled)
